(19) United States
(12) Patent Application Publication
(10) Pub. No.: US 2020/0354608 A1
(43) Pub. Date: Nov. 12, 2020

(54) AQUEOUS EMULSION OF BIOLOGICAL ANTIFREEZE PROTEIN FOR ROAD ANTI-ICING AND DEICING AND METHOD FOR MAKING SAME

(71) Applicant: Guangxi University, Nanning (CN)

(72) Inventors: Yongjun MENG, Nanning (CN); Qixiong ZHAO, Nanning (CN); Lishan LING, Nanning (CN); Junyi SHI, Nanning (CN); Jing LI, Nanning (CN); Junlin LIANG, Nanning (CN); Hongliu RONG, Nanning (CN)

(21) Appl. No.: 16/820,229

(22) Filed: Mar. 16, 2020

(30) Foreign Application Priority Data
May 10, 2019 (CN) 201910387388.8

(51) Int. Cl. C09D 189/00; C07K 14/435; C07K 14/415; C07K 14/46

(52) U.S. Cl. CPC C09D 189/00; C07K 14/461; C07K 14/415; C07K 14/43563

(57) ABSTRACT

This invention relates to road deicing, and particularly discloses an aqueous emulsion of biological antifreeze protein for road anti-icing and deicing and a method of preparing the same. The emulsion is prepared from 3.2-12.8% by weight of a biological antifreeze protein; 45-75% by weight of water; 0.2-0.8% by weight of a cationic emulsifier; 1-4% by weight of nitrile latex; and 8-22% by weight of phosphate-buffered saline. The aqueous emulsion prepared herein involves cheap raw materials, simple production processes, good deicing performance, displaying a complete freezing inhibition at −2° C. to 0° C. and a freezing probability of 15% or less at −4° C. to −2° C. The aqueous emulsion can also effectively reduce ice crystal size, having a brilliant application prospect in road anti-icing and deicing in winter.

### CROSS-REFERENCE TO RELATED APPLICATIONS

This application claims the benefit of priority from Chinese Patent Application No. 201910387388.8, filed on May 10, 2019. The content of the aforementioned application, including any intervening amendments thereto, is incorporated herein by reference in its entirety.

### TECHNICAL FIELD

The present application relates to road deicing, and more particularly to an aqueous emulsion of biological antifreeze protein for road anti-icing and deicing and a method for making the same.

### BACKGROUND OF THE INVENTION

Currently, in winter, road deicing is mainly performed by spraying a certain amount of salts, such as sodium chloride, on icy roads. However, this passive deicing method consumes a large amount of salt, and the hidden icy roads cannot be treated with the salts, resulting in unsatisfactory deicing.

Researchers at home and abroad have undertaken extensive research on methods of deicing road. In the recent decade, a silane-based hydrophobic coating, which imitates superhydrophobic behavior of organism such as lotus and applies surface wettability theory, is prepared and sprayed on road surfaces to form a super-hydrophobic and anti-icing film, thereby effectively preventing ice reforming on the roads. This ice removing method is in an active operational manner instead of the passive manner. However, this active method has complicated coating-manufacturing procedures and the coating prepared has poor durability.

In summary, there is an urgent need to develop an excellent deicing material.

### SUMMARY OF THE INVENTION

An object of this invention is to provide an aqueous emulsion of biological antifreeze protein for road anti-icing and deicing and a method of preparing the same to overcome technical problems in the prior art.

The invention adopts the following technical solutions to reduce traffic damage caused by road icing.

In a first aspect, the invention provides the aqueous emulsion of biological antifreeze protein for road anti-icing and deicing, comprising:

3.2-12.8% by weight of a biological antifreeze protein;

45-75% by weight of water;

0.2-0.8% by weight of a cationic emulsifier;

1-4% by weight of nitrile latex; and

8-22% by weight of phosphate-buffered saline.

In some embodiments, the biological antifreeze protein is selected from the group consisting of a yellow mealworm antifreeze protein, a beetle antifreeze protein, an ammopiptanthus mongolicus antifreeze protein, an ophiopogogon japonicus antifreeze protein, a yellow grouper antifreeze protein and a combination thereof. The biological antifreeze protein lowers the freezing point of water on road surfaces, reduces the ice crystal size and inhibits the growth of ice crystals due to its own effects of thermal hysteresis and ice recrystallization inhibition.

The cationic emulsifier is classified as cationic, and the cationic emulsifier is specifically an EL-20 emulsion polymerization emulsifier.

The phosphate-buffered saline is used to adjust a resulting mixed solution to pH 4-8 and ensure the activity of antifreeze protein.

The invention has the following advantages for this first technical solution.

(1) The biological antifreeze protein plays a key role in anti-freezing, and the rest ingredients in the aqueous emulsion are carriers which are adhered to the biological antifreeze protein. The biological antifreeze protein is used to lower the freezing point of water on surfaces of emulsified asphalt roads and reduce the ice crystal size.

(2) The cationic emulsifier is used to make a positively charged polymer emulsion, so that the polymer emulsion can be normally used in hard water or acidic conditions.

(3) The nitrile latex can be used to improve the bonding performance of aqueous emulsion, and it has a uniform molecular weight distribution and a uniform particle size distribution, as well as resistance to oil, acid and alkali.

(4) The phosphate-buffered saline maintains the activity of antifreeze protein, salt-balance and a suitable pH.

(5) The interaction between the cationic emulsifier, nitrile latex and the phosphate-buffered saline allows the biological antifreeze protein for better resistance to freezing and stability.

In a second aspect, the invention further provides a method for preparing the aqueous emulsion of biological antifreeze protein for road anti-icing and deicing, comprising:

(1) preparing 3.2-12.8% by weight of the biological antifreeze protein, 45-75% by weight of water, 0.2-0.8% by weight of the cationic emulsifier, 1-4% by weight of nitrile latex and 8-22% by weight of the phosphate-buffered saline;

(2) heating the water to 60-80° C.;

(3) adding the cationic emulsifier to the heated water and uniformly dispersing the cationic emulsifier in the water; and adding the phosphate-buffered saline to adjust the mixed solution to pH 4-8;

(4) transferring the mixed solution obtained in step (3) to a high shear emulsifier and adding the nitrile latex to the high shear emulsifier followed by rotating and stirring at 800-1000 rpm for 3-5 min to obtain an aqueous emulsion; and transferring the aqueous emulsion to a container; and

(5) cooling the aqueous emulsion obtained in step (4) to room temperature followed by adding the biological antifreeze protein; and stirring the resulting mixture uniformly, thereby obtaining a final product.

The invention has the following advantages for this second technical solution.

(1) The aqueous emulsion of biological antifreeze protein prepared herein involves cheap raw materials, simple production processes, and simple and easy construction.

(2) The aqueous emulsion of biological antifreeze protein prepared herein has good deicing performance, displaying a complete freezing inhibition at −2° C. to 0° C. and a freezing incidence of 15% or less at −4° C. to −2° C.

(3) The aqueous emulsion of biological antifreeze protein prepared herein is biodegradable and environmental friendly, having a brilliant application prospect in road anti-icing and deicing in winter.

The invention further provides a use method of the aqueous emulsion of biological antifreeze protein for road anti-icing and deicing, comprising:

cleaning the road surfaces followed by uniformly spraying the aqueous emulsion of biological antifreeze protein using an aqueous emulsion-spraying vehicle.

The aqueous emulsion of biological antifreeze protein has ease of use, since the traffic can be resumed soon after the spraying.

Deicing mechanism of the invention is described as follows.

The aqueous emulsion of biological antifreeze protein prepared in the invention is sprayed on surfaces of concrete roads or asphalt roads and is rolled by vehicles driving on the roads. During the rolling, the biological antifreeze protein in the aqueous emulsion is effectively released due to lifting force of tires applied on the roads. At the same time, the nitrile latex in the invention has resistance to oil, solvent and chemicals and has good compatibility with polar polymer materials to effectively prevent the loss of biological antifreeze proteins and ensure the sustained release of the biological antifreeze protein in the emulsion, resulting in long-lasting road anti-icing performance.

### DETAILED DESCRIPTION OF EMBODIMENTS

This invention will be further described below with reference to the embodiments. However, these embodiments are not intended to limit the present invention. Any modifications and changes made by those skilled in the art without departing from the technical solutions of the invention shall fall within the scope of the claims of the invention. The amount of materials are represented by parts by weight, and in the following description of embodiments, one part by weight corresponds to one kilogram, for the purposes of illustration.

### EXAMPLE 1

An aqueous emulsion of biological antifreeze protein for road anti-icing and deicing was prepared from 3.2 kg of a yellow mealworm antifreeze protein, 45 kg of water, 0.2 kg of a cationic emulsifier, 1 kg of nitrile latex and 22 kg of phosphate-buffered saline.

In this embodiment, a potassium phosphate-buffered solution (pH=1) was employed as the phosphate-buffered saline, and the final product antifreeze protein emulsion prepared herein had a pH of 4.

The aqueous emulsion of biological antifreeze protein for road anti-icing and deicing was prepared as follows.

(1) 3.2 kg of the yellow mealworm antifreeze protein, 45 kg of water, 0.2 kg of the cationic emulsifier, 1 kg of nitrile latex and 22 kg of the phosphate-buffered saline were prepared for use.

(2) The water was heated to 60-80° C.

(3) The cationic emulsifier was added to the heated water and uniformly dispersed in the water. The phosphate-buffered saline was added to adjust a pH of the mixed solution.

(4) The mixed solution obtained in step (3) was transferred to a high shear emulsifier, and the nitrile latex was added to high shear emulsifier followed by rotating and stirring at 800-1000 rpm for 3-5 min to obtain an aqueous emulsion. The aqueous emulsion was then transferred to a container.

(5) The aqueous emulsion obtained in step (4) was cooled to room temperature followed by adding the biological antifreeze protein. The resulting mixture was uniformly stirred, thereby obtaining a final product.

### EXAMPLE 2

An aqueous emulsion of biological antifreeze protein for road anti-icing and deicing was prepared from 12.8 kg of an ammopiptanthus mongolicus antifreeze protein, 75 kg of water, 0.8 kg of a cationic emulsifier, 4 kg of nitrile latex and 22 kg of phosphate-buffered saline.

In this embodiment, a potassium phosphate-buffered solution (pH=12) was employed as the phosphate-buffered saline, and the final product antifreeze protein emulsion prepared herein had a pH of 8.

The preparation method was the same as that in Example 1.

### EXAMPLE 3

An aqueous emulsion of biological antifreeze protein for road anti-icing and deicing was prepared from 8 kg of a yellow grouper antifreeze protein, 55 kg of water, 0.6 kg of a cationic emulsifier, 2 kg of nitrile latex and 8 kg of phosphate-buffered saline.

In this embodiment, a potassium phosphate-buffered solution (pH=4.5) was employed as the phosphate-buffered saline, and the final product antifreeze protein emulsion prepared herein had a pH of 6.

The preparation method was the same as that in Example 1.

### EXAMPLE 4

An aqueous emulsion of biological antifreeze protein for road anti-icing and deicing was prepared from 4 kg of a yellow grouper antifreeze protein, 4 kg of an ophiopogogon japonicus antifreeze protein, 45 kg of water, 0.2 kg of a cationic emulsifier, 1 kg of nitrile latex and 15 kg of phosphate-buffered saline.

In this embodiment, a potassium phosphate-buffered solution (pH=3) was employed as the phosphate-buffered saline, and the final product antifreeze protein emulsion prepared herein had a pH of 5.

The preparation method was the same as that in Example 1.

### EXAMPLE 5

An aqueous emulsion of biological antifreeze protein for road anti-icing and deicing was prepared from 1.6 kg of a yellow grouper antifreeze protein, 1.6 kg of an ophiopogogon japonicus antifreeze protein, 55 kg of water, 0.6 kg of a cationic emulsifier, 2 kg of nitrile latex and 22 kg of phosphate-buffered saline.

In this embodiment, a potassium phosphate-buffered solution (pH=1) was employed as the phosphate-buffered saline, and the final product antifreeze protein emulsion prepared herein had a pH of 4.

The preparation method was the same as that in Example 1.

### EXAMPLE 6

An aqueous emulsion of biological antifreeze protein for road anti-icing and deicing was prepared from 6.4 kg of a yellow grouper antifreeze protein, 6.4 kg of an ophiopogogon japonicus antifreeze protein, 55 kg of water, 0.6 kg of a cationic emulsifier, 2 kg of nitrile latex and 10 kg of phosphate-buffered saline.

In this embodiment, a potassium phosphate-buffered solution (pH=6.7) was employed as the phosphate-buffered saline, and the final product antifreeze protein emulsion prepared herein had a pH of 7.

The preparation method was the same as that in Example 1.

### COMPARATIVE EXAMPLE 1

An emulsion was prepared from 8 kg of a yellow grouper antifreeze protein, 8 kg of an ophiopogogon japonicus antifreeze protein, 35 kg of water, 1 kg of a cationic emulsifier, 0.5 kg of nitrile latex and 26 kg of phosphate-buffered saline.

In this embodiment, a potassium phosphate-buffered solution (pH=1) was employed as the phosphate-buffered saline, and the final product antifreeze protein emulsion prepared herein had a pH of 3.

The preparation method was the same as that in Example 1.

### COMPARATIVE EXAMPLE 2

A comparative product was prepared from 8 kg of a yellow grouper antifreeze protein and 55 kg of water.

The preparation method included the steps as follows.

The yellow grouper antifreeze protein was added to the water at room temperature followed by uniformly stirring. The final product was prepared for immediate use.

In Examples 1-6 and Comparative Examples 1 and 2, the yellow mealworm antifreeze protein, the beetle antifreeze protein, the ammopiptanthus mongolicus antifreeze protein, the ophiopogogon japonicus antifreeze protein, and the yellow grouper antifreeze protein could be obtained from the corresponding living body through sampling, grinding, ion exchange chromatography, gel filtration, extraction and purification.

### Deicing Effect Evaluation

Sodium chloride, the aqueous emulsions prepared in Examples 1-6 and the emulsions prepared in Comparative Examples 1 and 2 had undergone freezing experiments at 0° C. to −2° C. and −2° C. to −4° C., respectively for 2 h. The results were shown in Table 1.

TABLE 1
Freezing probability results

| | Freezing probability(%) | Freezing probability(%) |
|---|---|---|
| | at 0° C. to −2° C. | at −2° C. to −4° C. |
| Sodium | 4.7 | 18.8 |
| Chloride | | |
| Example 1 | 0 | 10.4 |
| Example 2 | 0 | 9.1 |
| Example 3 | 0 | 13.7 |
| Example 4 | 0 | 12.5 |
| Example 5 | 0 | 14.6 |
| Example 6 | 0 | 11.3 |
| Comparative | 4.3 | 16.5 |
| Example 1 | | |
| Comparative | 5.6 | 20.6 |
| Example 2 | | |

It can be seen from Table 1 that the aqueous emulsions of Examples 1-6 had the same freezing probability of 0 at 0° C. to −2° C., and sodium chloride had a freezing probability of 4.7% at 0° C. to −2° C. The aqueous emulsions of Examples 1-6 at −2° C. to −4° C. have a 4.2-9.7% lower freezing probability than sodium chloride, while the emulsions of Comparative Examples 1 and 2 at −2° C. to −4° C. have a 1.9-11.5% greater freezing probability. From which, it could be concluded that the aqueous emulsions of Examples 1-6 had better deicing performance than sodium chloride and the emulsions of Comparative Examples 1 and 2, being able to effectively improve the friction coefficient of icy roads and avoid the traffic accidents caused by road icing.

### Stability Evaluation:

The aqueous emulsions prepared in Examples 1-6 and the emulsion prepared in Comparative Example 1 were left standing at room temperature for stability tests. The emulsion state results as observed were shown in Table 2.

TABLE 2
Emulsion state results

| | Day 7 | Day 15 | Day 30 |
|---|---|---|---|
| Example 1 | Stable, without | Stable, without | Stable, without |
| | layering | layering | layering |
| Example 2 | Stable, without | Stable, without | Stable, without |
| | layering | layering | layering |
| Example 3 | Stable, without | Stable, without | Stable, without |
| | layering | layering | layering |
| Example 4 | Stable, without | Stable, without | Stable, without |
| | layering | layering | layering |
| Example 5 | Stable, without | Stable, without | Stable, without |
| | layering | layering | layering |
| Example 6 | Stable, without | Stable, without | Stable, without |
| | layering | layering | layering |
| Comparative | Stable, without | Layered | Layered |
| Example 1 | layering | | |

It can be seen from Table 2 that the emulsions of Examples 1-6 were relatively stable at room temperature, whereas the emulsion of Comparative Example 1 was layered after standing for a long time, indicating that the aqueous emulsions prepared in Examples 1-6 had good stability.

What is claimed is:

1. An aqueous emulsion of biological antifreeze protein for road anti-icing and deicing, comprising:
3.2-12.8% by weight of a biological antifreeze protein;
45-75% by weight of water;
0.2-0.8% by weight of a cationic emulsifier;
1-4% by weight of nitrile latex; and
8-22% by weight of phosphate-buffered saline.

2. The aqueous emulsion of claim 1, wherein it has a pH of 4-8.

3. The aqueous emulsion of claim 1, wherein the biological antifreeze protein is selected from the group consisting of a yellow mealworm antifreeze protein, a beetle antifreeze protein, an ammopiptanthus mongolicus antifreeze protein, an ophiopogogon japonicus antifreeze protein, a yellow grouper antifreeze protein and a combination thereof.

4. The aqueous emulsion of claim 1, wherein the nitrile latex has a uniform molecular weight distribution and a uniform particle size distribution, and has resistance to oil, acid and alkali; and the phosphate-buffered saline maintains the activity of antifreeze protein, salt-balance and a suitable pH.

5. A method for preparing the aqueous emulsion of claim 1, comprising:
(1) preparing 3.2-12.8% by weight of the biological antifreeze protein, 45-75% by weight of water, 0.2-0.8% by weight of the cationic emulsifier, 1-4% by weight of nitrile latex and 8-22% by weight of the phosphate-buffered saline;
(2) heating the water to 60-80° C.;
(3) adding the cationic emulsifier to the heated water and uniformly dispersing the cationic emulsifier in the water; and adding the phosphate-buffered saline to adjust the mixed solution to pH 4-8;
(4) transferring the mixed solution obtained in step (3) to a high shear emulsifier and adding the nitrile latex to the high shear emulsifier followed by rotating and stirring at 800-1000 rpm for 3-5 min to obtain an aqueous emulsion; and transferring the aqueous emulsion to a container; and
(5) cooling the aqueous emulsion obtained in step (4) to room temperature followed by adding the biological antifreeze protein; and stirring the resulting mixture uniformly, thereby obtaining a final product.
